(19) United States
(12) Patent Application Publication
(10) Pub. No.: US 2009/0054800 A1
(43) Pub. Date: Feb. 26, 2009

(54) Method and Device for Representing A Dynamic Functional Image of the Brain, By Locating and Discriminating Intracerebral Neuroelectric Generators and Uses Thereof

(75) Inventors: Jacques Martinerie, Palaiseau (FR); Sylvain Baillet, Velizy-Villacoublay (FR); Line Garnero, Clamart (FR); Jean-Philippe Lachaux, Lyon (FR); Michel Le Van Quyen, Paris (FR); Bernard Renault, Paris (FR)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CENTRE NATIONAL DE LA RECHERCHE SCIENTIFIQUE-CNRS-, Paris Cedex 16 (FR)

(21) Appl. No.: 11/996,367

(22) PCT Filed: Jul. 10, 2006
(86) PCT No.: PCT/FR06/01679

§ 371 Date: Jul. 15, 2008

(30) Foreign Application Priority Data
Jul. 22, 2005 (FR) 0507848

(51) Int. Cl. A61B 5/04

(52) U.S. Cl. 600/544

(57) ABSTRACT

The invention relates to a method of representing a dynamic functional image of the brain. It consists in acquiring (A) for a specific duration a plurality of electrophysiological signals {esᵢ}₁^{N} of cerebral activity from a set of electrodes {Eᵢ}₁^{N} placed on the scalp (S) of the subject, in locating (B) the set of neuroelectric generators {\vec{g}ⱼₖ}₁₁^{JK} in the cerebral volume from a three-dimensional image {Cₖ}₁^{K} made up of successive cross-sections of the brain, and in applying the inverse problem, and within active zones that include neuroelectric generators discriminating (C) the amount of synchrony that exists between electrophysiological signal and neuroelectric generator pairs in a plurality of frequency bands, to detect groups of discriminating neural networks {RNdₖ}₁^{K}. The invention is useful for non-invasive study of provoked or unprovoked functional anomalies.

The present invention relates to a method and to a device for representing a dynamic functional image of the brain by locating and discriminating intracerebral neuroelectric generators, and it also relates to uses thereof.

In a person, any cerebral act is the result of cooperation between a number of neural networks spatially distributed in the intracerebral space in a functional network.

At present, despite recent advances, the main cerebral imaging techniques such as EEG (electroencephalography), MEG (magnetoencephalography), fMRI (functional magnetic resonance imaging), and PET (positron emission tomography) can provide maps only of cerebral activation areas, but without enabling account to be taken directly of interactions between these areas and these activators.

Characterizing these functional networks requires identifying the cerebral areas involved, understanding the interaction mechanisms between them, and precise quantification of those interactions.

It is not possible to observe the operation of the aforementioned neural networks based only on cerebral activity mapping produced by the aforementioned imaging techniques.

Amongst all of the cerebral areas that are simultaneously active, it is not possible to discriminate those that are participating in the same functional network, since merely observing that areas are simultaneously active is not sufficient to conclude that those areas are engaged in the same functional, pathological, or cognitive process.

All currently known approaches with a comparable aim are based on the idea that the existence of a coupling between two intracerebral areas must be reflected in a correlation between their neuroelectric activities.

The activity of a group of neurons, for example a cortical column, can be characterized by two types of physiological measurements:

1) temporal coding by the rate of neural discharges per second; or 2) coding of synchronization of oscillatory activities of the cerebral areas involved in the same functional network.

Work has been done on applications of the second of the above types of physiological measurements.

A first application is the subject of U.S. Pat. Nos. 6,442,421 and 6,507,754 granted to M. LE VAN QUYEN, J. MARTINERIE, F. VARELA, and M. BAULAC.

That application relates essentially to a method and a device for anticipating epileptic seizures based on a surface electroencephalogram.

A second application is the subject of French Patent Application FR 2 845 883 in the name of CNRS (Center National de la Recherche Scientifique).

This second application relates to characterizing cognitive states on the basis of surface encephalograms.

The above application proves satisfactory. However, it would appear to be limited in that it is essentially based on a process of statistically validating a period of real-time analysis.

An object of the present invention is to provide a method and a device making it possible to establish a true representation of a dynamic functional image of the brain by locating and discriminating intracerebral neuroelectric generators in the intracerebral space as a whole.

Another object of the present invention is to provide a method and a device making it possible to establish a plurality of dynamic functional images of the brain, whereby one or more of those dynamic functional images can be associated with the same functional, pathological, or cognitive process.

A further object of the present invention is to provide a method and a device making it possible to establish one or more dynamic functional images of the brain, making it possible to characterize both time information and spatial information relating to the neuroelectric activity of cerebral activity areas forming a functional network.

A further object of the present invention is in particular to provide a method and a device for representing a dynamic functional image of the brain making it possible to provide true non-invasive imaging of the functional integration and functional connectivity of cerebral areas in a functional, pathological, or cognitive process state.

A further object of the present invention is in particular to provide a tool based on the method and the device of the invention making it possible to characterize signatures of substances, drugs, or medicines generating one or more provoked or unprovoked functional anomaly states of the brain, the signatures being represented in the form of dynamic functional images.

Finally, a further object of the present invention is to provide a tool based on the method and the device of the invention making it possible to characterize signatures of specific cognitive states such as vigilance, diffuse attention, sleepiness, etc., the signatures being represented in the form of dynamic functional images.

The method of the invention for representing a dynamic functional image of the brain by locating and discriminating intracerebral neuroelectric generators is noteworthy in that it consists at least, during a particular recording time, in acquiring a plurality of electrophysiological signals emitted and/or induced by cerebral activity from a plurality of electrodes spread out substantially over the scalp of the cranium protecting the brain, and in digitizing said electrophysiological signals in order to constitute a cerebral activity analysis database, in locating all the neuroelectric generators in the cerebral volume by acquiring an electronic map of the positions of the electrodes from a three-dimensional image of the brain made up of successive sections, and in recording electrophysiological signals on those electrodes. Application of the inverse problem, based on segmentation of the cerebral cortex obtained from the three-dimensional brain image, the electrophysiological signals, and the electronic map of the location, enables the spatial locations of the intracerebral neuroelectric generators to be determined and makes it possible from amongst the active areas including neuroelectric generators to compute the amount of synchrony that exists between any of the pairs of said neuroelectric generators. This quantification is effected for a plurality of frequency bands in order to detect groups of discriminating neural networks and to constitute a database of reference states representing the dynamic functional image.

The method of the invention is also noteworthy in that, for a dynamic functional image acquired during a particular recording time, it further consists in matching the functional image with one class of functional images from a plurality of functional images, each class of said plurality of classes of functional images characterizing a cerebral state of the brain of the subject.

The dynamic functional image of the brain that is obtained using the invention is noteworthy in that it includes a three-dimensional image of said brain made up of successive sections each representing an individual image of said brain and, in at least one individual image, at least one neuroelectric generator of neuroelectric signals represented by a marker, each neuroelectric generator being characterized in terms of its position in said individual image, in terms of its electric current density, and in terms of its neuroelectric signal emission direction, all neuroelectric generators of a current individual image adjoining a neuroelectric generator of a preceding and/or subsequent individual image and having substantially the same neuroelectric signal emission direction and synchrony over a particular consistency time constituting a group of neural networks discriminating functional states representing said dynamic functional image of the brain.

The device of the invention for representing a dynamic functional image of the brain is noteworthy in that it comprises at least a circuit for acquiring during a particular recording time a plurality of electrophysiological signals emitted and/or induced by cerebral activity from a plurality of electrodes spread out substantially over the scalp of the cranium protecting the brain, and for storing and backing up these electrophysiological signals to constitute a cerebral activity analysis database, a circuit for acquiring a three-dimensional image of the brain made up of successive sections, a module for computing the location of all the neuroelectric generators of the intracerebral neuroelectric signals in the intracerebral volume from the locations of the electrodes, from the three-dimensional image of said brain, from segmentation of the cerebral cortex, and for computing the application of the inverse problem, and a module for discriminating amongst the active areas that include neuroelectric generators, the amount of synchrony that exists between pairs of neuroelectric generators in a plurality of frequency bands in order to detect groups of discriminating neural networks and to construct a database of reference states representing said dynamic functional image.

The method and the device of the invention find uses in the non-invasive functional study of the human brain in the most diverse situations such as, in particular, the study of functional anomalies whether provoked or not by ingesting drugs, medicines, categorizing functional and/or clinical states, and relating them in a rational manner to specific pathological or cognitive states.

They can be understood better on reading the following description and examining the drawings, in which:

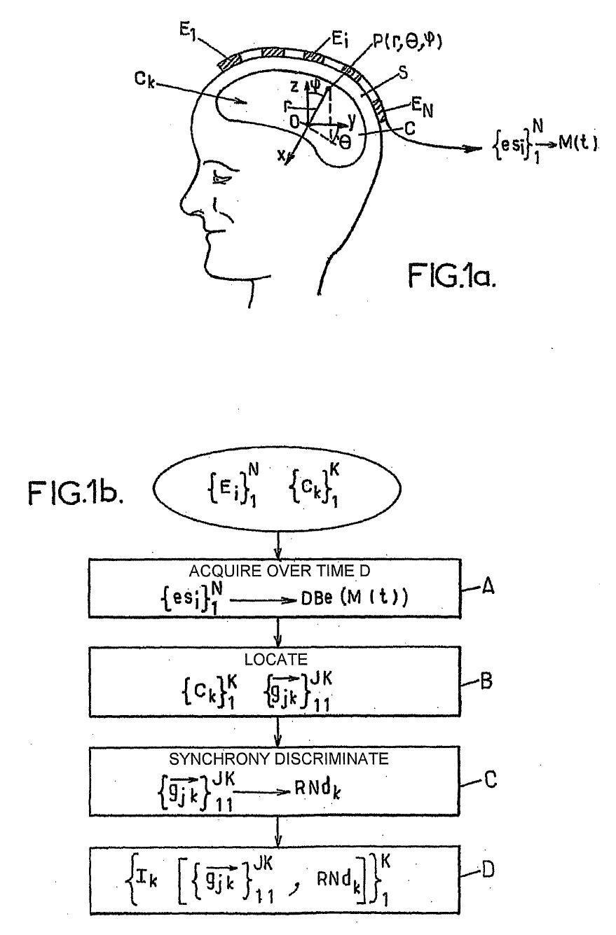

FIG. 1a is an illustrative representation in section in a vertical plane of symmetry of the entire head of a subject whose scalp is fitted with a network of electrodes in order to enable the method of the invention to be implemented;

FIG. 1b is a flowchart of the essential steps of implementing the method of the invention under the conditions illustrated in FIG. 1a;

FIG. 1c is an illustrative representation of a succession of individual dynamic functional images constituting a dynamic functional image in accordance with the present invention showing groups of discriminating neural networks constituting a cerebral activity area forming a functional network;

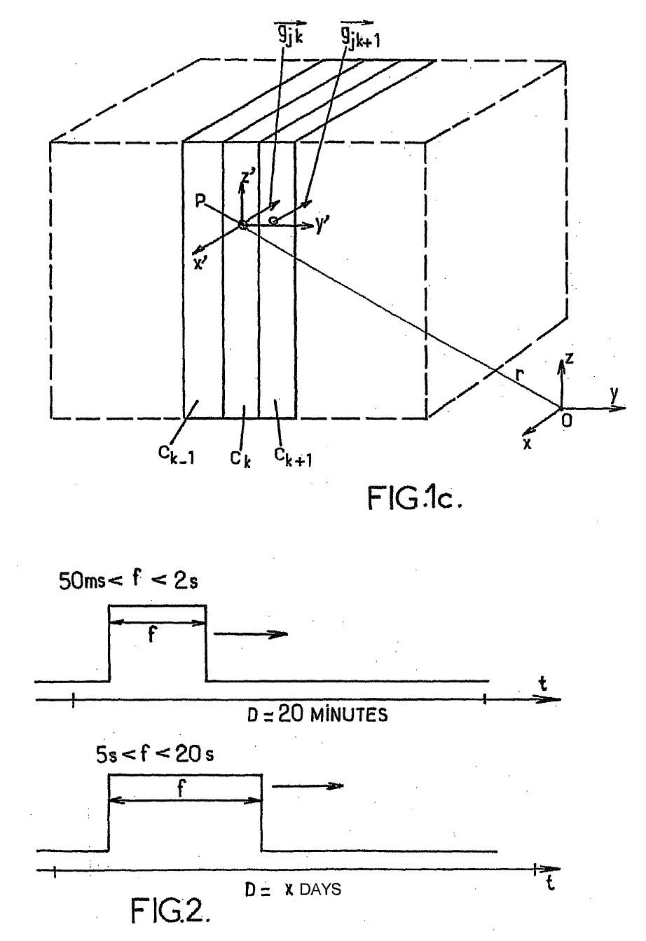

FIG. 2 is a timing diagram showing the implementation of a window for recording and analyzing electrophysiological signals, the recording time and the duration of the window being parameters set as a function of the chosen functional image class with a view to characterizing the cerebral state of the brain of the subject;

FIG. 3 represents, by way of illustration, a detail of the implementation of the step represented in FIG. 1b for locating all the neuroelectric generators in the cerebral volume;

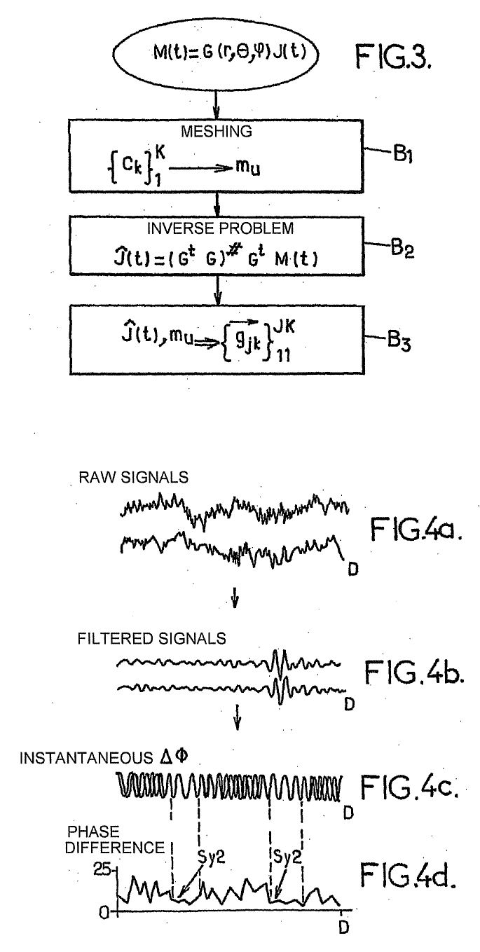

FIG. 4a is a timing diagram of raw EEG-type signals delivered by a pair of electrodes placed on the scale of a subject for a particular recording time;

FIG. 4b is a timing diagram of the signals from FIG. 4a after filtering;

FIG. 4c shows the phase difference obtained by spectrum analysis of the signals shown in FIG. 4b;

FIG. 4d shows the signal representative of variation in the phase difference between the signals shown in FIG. 4c over the recording time, revealing synchrony between these signals over certain ranges of the recording time;

FIG. 5 is a specific functional image of a brain showing the neuroelectric generators associated with the fingers of the right hand of a normal subject;

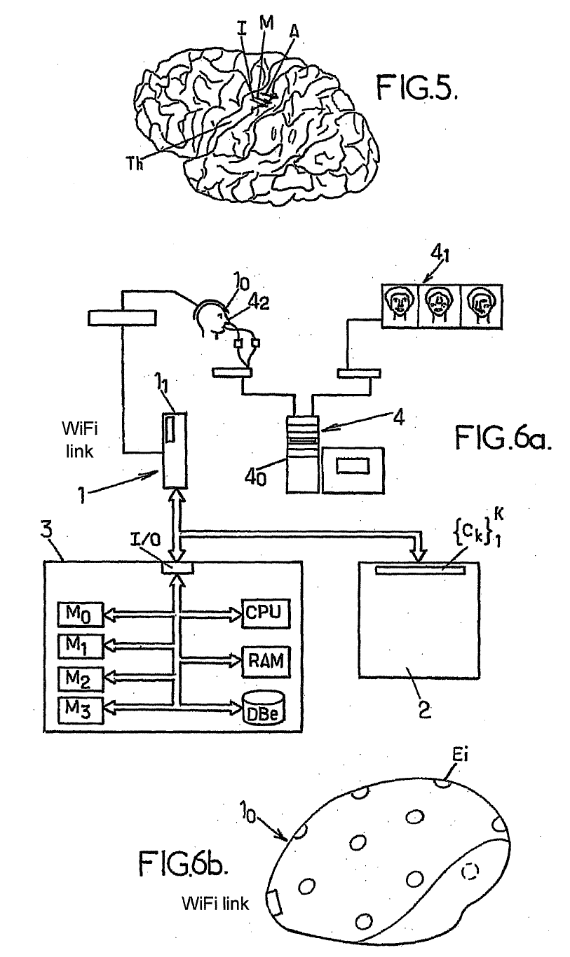

FIG. 6a is, by way of illustration, a functional block diagram of a device of the invention for representing a dynamic functional image of the brain; and

FIG. 6b shows a flexible cap fitted with electrodes for acquiring electrophysiological signals.

The method of the invention for representing a dynamic functional image of the brain is described below with reference to FIGS. 1a, 1b, and the subsequent figures.

FIG. 1a is a view in section in a vertical plane of symmetry showing the entire head of a subject for whom the method of the invention is applied.

The section plane shown is chosen by way of non-limiting example, and any section plane other than this one could be used.

As shown in FIG. 1a, Cₖ designates the section of the brain C and the entire head in the aforementioned section plane, this section consequently being represented in the plane of FIG. 1a.

The head of the subject, and in particular the scalp S, is equipped with a plurality of electrodes distributed over the scalp S of the cranium protecting the brain C. For example, the plurality of electrodes {Eᵢ}₁^{N} comprises N electrodes spread out in substantially regular manner over the scalp of the subject.

For example, as shown in FIG. 1a, O designates an arbitrary reference point situated in the section plane Cₖ and Oxyz designates a given system of axes for identifying any point P of the brain C by its polar coordinates r, θ, φ, relative to that system of axes.

It is therefore clear that, to implement the method of the invention, each electrode Eᵢ picks up an electrophysiological signal esᵢ of the EEG and/or MEG type in order to enable the method of the present invention to be implemented.

Referring to FIG. 1b, and in the light of the description given with reference to FIG. 1a, the method of the invention is noteworthy in that it includes acquiring a plurality of electrophysiological signals {esᵢ}₁^{N} during a step A and during a particular recording time D.

These electrophysiological signals are emitted and/or induced by the cerebral activity of the brain C and are picked up from the plurality of electrodes {Eᵢ}₁^{N}. These electrophysiological signals are digitized to constitute a cerebral activity analysis database DBe and the storage system is denoted M(t).

With regard to the nature of the aforementioned electrophysiological signals esᵢ, note that, in addition to signals generated directly by cerebral activity, as mentioned above, additional signals can be acquired simultaneously, and can consist in signals generated by movement of the eyes of the subject, cardiac activity signals, or any other electrophysiological signal that might be stored during the recording time.

All these signals are then organized as mentioned above to constitute the database DBe.

As represented in FIG. 1b, the step A is then followed by a step B of locating the set of neuroelectric generators within the cerebral volume corresponding to the cerebral activity of the subject.

This is advantageously effected on the basis of acquiring the electronic map of the position, of the electrodes {Eᵢ}₁^{N} placed on the scalp of the patient, as shown in FIG. 1a, and a three-dimensional image of the brain C made up of a set {Cₖ}, of successive sections.

It is clear in particular that, given the known positions of the acquisition electrodes Eᵢ, and, of course, the three-dimensional image of the brain C formed by the set {Cₖ}₁^{K} of sections, there is obtained a segmentation of the cerebral cortex, as is described below, with the positions of the electrodes being located on that model.

All the neuroelectric generators in the cerebral volume are then located by application of the inverse problem, which is defined as obtaining the local current densities in the cerebral cortex and, in particular, segmenting the cerebral cortex on the basis of the voltage measurements M(t) obtained from the electrophysiological signals esᵢ delivered by the set {Eᵢ}₁^{N} of electrodes.

It is therefore clear that applying the inverse problem makes it possible to determine the electronic map of the locations of the electrodes from the set {esᵢ}₁^{N} of electrophysiological signals and, of course, the spatial location of the neuroelectric generators of the intracerebral neuroelectric signals from the three-dimensional image of the brain made up of successive sections that provide a segmentation of the cerebral cortex.

In FIG. 1b, in step B, {\vec{g}_{jk}}₁₁^{JK} denotes the set of intracerebral neuroelectric signal generators.

It is clear, in particular, that each neuroelectric generator of intracerebral neuroelectric signals is defined not only in amplitude, i.e. in local current density, but also in orientation at each point P(r, θ, φ) of the brain C as described above.

In accordance with the method of the invention, once step B has been executed, all of the intracerebral generators are available, for each time t, in each successive section of rank k, and therefore, finally, throughout the intracerebral volume.

As shown in FIG. 1a, step B is then followed by a step C of discriminating, among the active areas of the brain and in particular from each section Cₖ including neuroelectric generators, the amount of synchrony that exists between pairs of neuroelectric generators in a plurality of frequency bands in order to detect groups of discriminating neural networks constituting functional networks arising out of the cerebral activity of the subject.

In FIG. 1b, in step C, {gⱼₖ}₁₁^{JK}→RN_{dk} symbolically denotes this operation of discriminating synchrony.

In the above relationship, RN_{dk} designates the groups of discriminating neural networks corresponding to a functional network as mentioned above, for example for a section Cₖ.

Following execution of the aforementioned step C, and after completing execution of the process of the invention, i.e. in a step D, there is available a functional image that can be formed by individual dynamic functional images, each of which can correspond to one of the sections Cₖ having associated therewith at least one active neuroelectric generator \vec{g}ⱼₖ, and a group or part of a group of discriminating neural networks RN_{dk}. For this reason, {Iₖ[{\vec{g}ⱼₖ}₁₁^{JK}, RNdₖ]}₁^{K} denotes the individual functional image.

Each functional image can correspond to a projection or intersection of a set of individual dynamic functional images, each corresponding to one of the sections Cₖ, for example, on a representation plane that can have any orientation relative to the direction of the sections.

FIG. 1c shows a plurality of functional images formed by successive sections Cₖ₋₁, Cₖ, and Cₖ₊₁ in which different neuroelectric generators \vec{g}ⱼₖ are represented, each generator being located relative to the system of axes Oxyz as mentioned above, and each neuroelectric generator being defined in amplitude, i.e. in current density, and in orientation relative to a system of axes Px′y′z′ tied to the original system of axes.

Referring to FIG. 1c, a group of discriminating neural networks consists of a group of neuroelectric generators present in individual images and therefore in successive sections Cₖ₋₁, Cₖ, and Cₖ₊₁, these generators having a similar orientation and satisfying the synchrony criterion defined with reference to step C in FIG. 1b.

For each functional image acquired during a recording time D, the method of the invention matches the functional image to one of a plurality of classes of functional images, each class of that plurality of classes of functional images characterizing a cerebral state of the brain of the subject, as is described below.

Accordingly, referring to FIG. 2, the step of acquiring and processing a plurality of electro- physiological signals {esᵢ}₁^{N} is effected in real time with a maximum recording delay of less than 100 milliseconds.

Referring to the aforementioned FIG. 2, the recording time D is a parameter that can be set over a time range, the recording time lying between a minimum recording time of the order of 20 minutes for recording and representing a functional image of the brain relating to one or more cognitive states, and a recording time D of several days, denoted D=x days in FIG. 2, for recording and representing a functional image of the brain relating to one or more provoked or unprovoked functional anomaly states of the brain. Provoked anomaly states can be provoked by ingestion of drugs, medicines, or any other substance, for example accidental ingestion.

Clearly, and in particular given the maximum recording delay of less than 100 milliseconds, the electrophysiological signals esᵢ are recorded using a sampling frequency sufficient for this purpose.

Where the use of the stored data is concerned, that is to say the data M(t) referred to above and constituting the database DBe, the stored data can be used in the following manner, during the recording time as represented in FIG. 2, and between active areas, to discriminate the amount of synchrony that exists between pairs of electrophysiological signals from the neuroelectric generators.

The use of the aforementioned signals then consists in effecting this discrimination over a sliding time window whose duration f is from 50 milliseconds to 2 seconds (s) for representing a functional image of the brain relating to one or more cognitive states and over a sliding time window whose duration is from 5 s to 20 s for representing a functional image of the brain relating to one or more provoked or unprovoked functional anomaly states of the brain, as also represented in FIG. 2.

The step B of locating the neuroelectric generators {\vec{g}ⱼₖ}₁₁^{JK} is described in more detail below with reference to FIG. 3.

An explanation of the procedure is given first with reference to FIG. 3.

The discretization of the integral equations that govern computation of the scalp electrical potentials establishes an instantaneous linear relationship between the measurements M(t) and the amplitudes, i.e. the current densities of the neuroelectric generators distributed within the cerebral volume. In the presence of additive noise, the problem is therefore to estimate the distribution of the cortical currents or the current densities J from which the stored signals M(t) originate and thus to solve an inverse problem in the manner of many other image reconstruction applications in medical imaging, for example.

There is no single solution to the problem of estimating the sources, i.e. the neuroelectric generators of an electromagnetic field measured at the external surface of a conductive volume.

The problem is a fundamentally ill-stated problem in the J. Hadamard sense. The method of the invention therefore proposes to use an estimator that imposes controlled anatomical and electrophysiological constraints and guarantees that a unique estimate is obtained.

The corresponding estimator is described below with reference to FIG. 3.

Referring to FIG. 3, the set M(t)=G(r,θ,φ)J(t) of stored measurements is available, where:
M(t) designates the set of recordings obtained, i.e. the values of the electrophysiological signals in the form of values of electrical potentials on the surface of the scalp, for example;
G(r,θ,φ) designates the transfer matrix between the surface electrophysiological signals {esᵢ}₁^{N} present on the scalp at each local point of the intracerebral volume and the estimated corresponding local current density Ĵ (t).

As shown in FIG. 3, the locating step B entails executing a step B₁ consisting in applying the constraints stemming from the individual anatomy introduced by segmentation and surface meshing of the parenchyma.

This operation is based on the set {Cₖ}₁^{K} of successive sections enabling the aforementioned meshing mᵤ to be obtained.

The step B₁ is then followed by a step B₂ of computing the local current densities by solving the inverse problem in application of the following equation, in which λ is the regularization term and I is the identity matrix:

\hat{J} (t)=(G^{t}G)#G^{t}M(t)+λI

Following the step B₂ local current densities at a given time at any point in the intracerebral volume with coordinates r, θ, φ are therefore available.

In the above equation:
Ĵ (t) designates the estimate of the local current density;
G^{t} designates the transposed transfer matrix of the matrix G representing the transfer matrix G(r, θ, φ);
(G^{t}G)# designates the pseudo-inverse of the transfer matrix G.

Because of the current density value estimation speed constraints that apply to use of the method and the device of the invention, and assuming independent and identically distributed Gaussian noise, a solution that is satisfactory in terms of a compromise between spatial resolution and computation time is the solution that minimizes the energy of the residuals and the norm of the neural currents, the resulting estimator being an unbiased estimator with minimum norm in the least squares sense.

The step B₂ is then followed by a step B₃ of computing the positions of the functional parameters, i.e. the amplitude and orientation of the neuroelectric generators \vec{g}ⱼₖ, in the form of individual electric current sources over the meshing of the cortical surface.

In the step B₂ in FIG. 3, this operation is represented by the symbolic relationship:

\vec{J}(t), mᵤ →{\vec{g}ⱼₖ}₁₁^{JK}

Thus active areas are available, where each active area includes at least one neuroelectric generator.

Where execution of the step B₂ is concerned, note that the physical models involving the measurements M(t) rely on resolving Ohm's law in three dimensions. It is justifiable to neglect the electromagnetic field propagation phenomena at the physiological frequencies used. The corresponding modeling can then be effected either analytically in the context of the spherical geometry with the original system of axes, or numerically by considering the specific geometry of the envelopes of the bony tissue and of the scalp S.

One specific implementation of the synchrony discrimination step C described above with reference to FIG. 1b is described in more detail below with reference to FIGS. 4a to 4d.

Generally speaking, referring to the aforementioned figures, note that the step of discriminating the amount of synchrony that exists between pairs of neuroelectric generators in the active areas including neuroelectric generators in a frequency band consists at least in statistically evaluating the PLS synchronization between two signals from a pair of neuroelectric generators by means of the circular variance of the phase difference between those signals, or of the normalized Shannon entropy of that phase difference.

A theoretical justification is given below, before the description as such as given with reference to FIGS. 4a to 4d.

Generally speaking, the instantaneous phase of a signal can be computed with the aid of an analytical signal. The analytical signal concept was introduced by Gabor in 1946 and has recently been applied to experimental data.

Accordingly, with reference to the aforementioned concept, for an arbitrary signal s(t), i.e. for any stored electrophysiological signal M(t), the analytical signal z is a complex time-dependent function defined by the following equation:

ζ(t)=s(t)+\tilde{js} (t)=A(t)e^{jØ(t)} (1)

In the above equation, the function \tilde{j}\tilde{s}(t) is the Hilbert transform of s(t) in the form:

$$\tilde{s} ( t ) = \frac{1}{π} P . V . {\int }_{- ∞}^{+ ∞} \frac{s ( t )}{t - τ} d t \qquad ( 2 )$$

In the Hilbert transform, P.V. indicates that the integral is computed in the sense of the Cauchy principal value. The instantaneous amplitude A(t) and the instantaneous phase F(t) of the signal S(t) are uniquely defined by the above equation 1.

With reference to equation 2, \tilde{s}(t) is considered as the convolution product of the signal s(t) and 1/π.

Consequently, applying the Hilbert transform to the signal s(t) is equivalent to applying filtering with a unitary amplitude response and a phase response shifted by π/2 for all frequencies.

Although the aforementioned transform process can in theory be applied to signals with a wide frequency band, the phase concept is not very explicit in such circumstances and, in practice, only narrowband signals obtained by filtering are used.

Consequently, filtering is applied in a specific frequency band. A number of frequency bands can nevertheless be retained, but the same frequency band is used for two signals that are in 1:1 synchrony. Other frequency bands can be used to study n:m synchronies. The PLS synchrony between the two signals is statistically evaluated by means of two indices: the circular variance, and the phase difference between the signals or the normalized Shannon entropy of the phase difference.

The circular variance satisfies the equation:

$$VC = \vert {\sum }_{k = 1}^{M} {e}^{( i Δ Φ k )} \vert$$

and the normalized Shannon entropy satisfies the equation:

γ=(Hₘₐₓ−H)/Hₘₐₓ

In the latter equation, the entropy is defined by the equation:

$$H = {\sum }_{m = 1}^{M} {p}_{m} ln {p}_{m}$$

In the above equation:
M designates the number of phase value classes;
Hm=1n(M) designates the maximum entropy;
pₘ designates the relative frequency of the phase difference in the m^{th} phase value class;
1n designates the natural logarithm.

The optimal number of phase value classes is M=exp[0.626+0.41n(P−1)] where P designates the number of phase differences to be classified.

Given the introduction of the aforementioned normalization, the values of γ are from 0 (uniform distribution and no synchronization) to 1 (perfect synchronization).

The aforementioned computation is effected for all estimated source pairs or where appropriate, to reduce the computation time, by random or directed sampling.

For a number of sources, i.e. neuroelectric generators, equal to 27, the number of different pairs is 325, and for 64 generators it increases to 1953. It is impossible to use this procedure for a few hundred sources.

In practice, in the method of the invention, the real-time synchrony computation can advantageously be limited to 100 generators. Regions of interest for real-time processing are then chosen as a function of the experimental protocol adopted and the use of information-reducing statistical techniques (discriminatory analysis, spatial filters, etc.).

Accordingly, referring to the aforementioned FIGS. 4a to 4d and starting with the raw signals represented in FIG. 4a, for two signals constituting a pair stored over the recording time D the synchrony discrimination step C can consist, for example, in effecting filtering over a plurality of frequency bands to obtain the filtered signals shown in FIG. 4b, and then in performing the above-mentioned spectrum analysis to obtain the instantaneous phase differences between the aforementioned signals, as shown in FIG. 4c.

The above-mentioned statistical study based on circular variance indices of the phase difference between the signals or the normalized Shannon entropy of that phase difference can then be carried out to quantify the phase differences, as shown in FIG. 4b, in which the synchronies Sy1 and Sy2 can be highlighted, for a substantially minimum phase difference of constant relative value compared with other areas of the recording time.

Finally, referring to FIG. 4d, synchrony between pairs of neuroelectric generators can advantageously be established in terms of synchrony time ranges. This enables a temporal representation of the activity of the pairs of neuroelectric generators that produces a true dynamic functional image of the brain.

When the neuroelectric generators have been placed in the individual functional dynamic images, and in particular in a succession thereof as shown in FIG. 1c, the method of the invention can then be used to obtain any dynamic functional image of the brain, such as that shown in FIG. 5.

This kind of image includes as least one three-dimensional image of the brain consisting of successive sections, each representing an individual image of the brain as described with reference to FIG. 1c. In FIG. 5, the successive sections are not shown, in order not to overcomplicate the drawing.

Furthermore, as shown in FIG. 5, the dynamic functional image includes, in at least one of the individual images, and where applicable in several of them, at least one neuroelectric generator of intracerebral neuroelectric signals represented by a marker. In FIG. 5 the marker is an oriented arrow of amplitude that in fact represents the local current density at the point at which the corresponding neuroelectric generator is positioned and of orientation that corresponds exactly to the orientation in the original system of axes of the electric current generated by the neuroelectric generator.

Referring to FIG. 5, note that each neuroelectric generator is characterized in position in the individual image, and thus in the resulting dynamic functional image, in terms of the electric current density and the direction of emission of the corresponding neuroelectric signals.

It is therefore clear that each neuroelectric generator of a current individual image, near a neuroelectric generator of a preceding and/or subsequent individual image, as shown in FIG. 1c, and having substantially the same direction of emission of electric signals and a synchrony over a particular period of consistency, constitutes a group of neural networks discriminating functional states representative of the dynamic functional image of the brain.

FIG. 5 advantageously represents the neuroelectric generators associated with the fingers of the right hand of a normal subject, i.e. one who has no functional anomaly of the fingers of the hand, and consequently no corresponding brain functional anomaly of the brain.

Note in FIG. 5 that each finger is represented by a neuroelectric generator constituting an equivalent dipole. These oriented generators are perpendicular to the cortical surface and tangential to the surface of the head, and correspond to the activity of neural macrocolumns situated in the central sulcus represented in FIG. 5, in which the thumb Th is represented by the oriented arrow, the index finger I by a particular arrow, the middle finger M by another parallel arrow, and the ring finger A by a different parallel arrow.

Note that the neuroelectric generators associated with the fingers are represented in anatomical order with great accuracy.

It is clear, in particular, that the functional images produced by the method of the present invention enable immediate detection of any functional anomaly of cortical representation of the human body in the brain, which functional images can, of course, be divided into classes representative either of a state of absence of functional anomalies or, to the contrary, of a class of functional anomalies and subclasses corresponding to an anomaly of one of the fingers considered.

Allocating the dynamic functional images produced by the method of the invention into classes of a category of classes means that the method of the invention can be implemented with an aim of decision-oriented discrimination.

This applies in the example described above with reference to FIG. 5 in particular.

Thus for a given recording time D, for example one or several seconds, and for a particular synchrony of the electrophysiological signals esᵢ, it is then possible to assign the corresponding dynamic functional image obtained to a specific class characterizing one of a number of cerebral states.

The corresponding problem is that of classification and, of course, assumes the a priori definition of a set of classes as mentioned above with reference to FIG. 5.

This decision-oriented procedure must take account of all pairs of electrodes Eᵢ. Under these conditions, for a number N of electrodes equal to 100 and for 14 frequency bands determined by the filtering effected in the processing represented in FIGS. 4a to 4b, a classification variables space of dimension p=70700 is obtained.

In a large space, as indicated above, obtaining stable predictions is conditional on procedural constraints of working in a number of contiguous small spaces and using a multi-classifier strategy to take interactions between those spaces into account.

Thus a first sorting of variables is effected between the selected classes for all frequency bands, for example using a Fisher discrimination test, so as to retain only the best 300, for example.

Then, for all these latter variables and for each frequency band, LDA or SVM analysis is carried out and the boundaries between the classes are retained.

This kind of binary discrimination procedure, i.e. discrimination between two classes, as mentioned above with reference to FIG. 5, for example, reduces the space from 70700 dimensions to 300 dimensions and then to 14 dimensions, i.e. one dimension per frequency band used. The final classification over this reduced space is arrived at through a combination of multi-classifiers such as LDA, NN, or SVM.

A more detailed description of a device of the present invention for representing a dynamic functional image of the brain is described below with reference to FIGS. 6a and 6b.

Referring to FIG. 6a, note that the device of the invention includes resources 1 for acquiring, during a particular recording time, a plurality of electrophysiological signals emitted and/or induced by cerebral activity, namely the signals {Eᵢ}₁^{N} described above. These signals are acquired from a plurality of electrodes forming a cap 1₀ that in use is placed on the scalp of the subject so as to spread the electrodes Eᵢ out regularly over the cranium protecting the brain C.

As shown in FIG. 6a, the electrodes Eᵢ and the aforementioned cap can advantageously be connected, for example by a WiFi type connection, to an acquisition computer 11 for storing and backing up the electrophysiological signals to constitute a cerebral activity analysis database. That database DBe can be remotely sited from the acquisition computer 11, as described below.

As also shown in FIG. 6a, the device of the invention further includes a resource 2 for acquiring a three-dimensional image of the brain made up of successive sections, i.e. the set {Cₖ}hd 1^{K} of sections.

FIG. 6a shows the acquisition resource 2 as advantageously formed by a reader or receiver of electronic files networked to the acquisition computer 11 and to an auxiliary processor unit 3 that executes functions for computing the locations of the set of neuroelectric generators and discriminating, among the active areas that include the aforementioned neuroelectric generators, the amount of synchrony that exists between the pairs of neuroelectric generators, as described above.

It is therefore clear that the three-dimensional image acquisition resources provide access either to an external database managed by an entity responsible for the clinical treatment of the subject or to said entity by way of a very high capacity optical disk reader, for example of dual layer DVD type.

Where the processor unit 3 is concerned, note that it is also networked to the acquisition computer 11 and can therefore be sited remotely from the acquisition computer, which means that the acquisition system for a particular subject can be self-contained.

In particular, it is clear that when using the method and the device of the invention for tests and to produce dynamic functional images over recording times of several days, the cap 10 can be rendered independent of the acquisition computer 11 by means of the indicated WiFi type connection, and that the acquisition computer 11 can consist of a laptop computer networked to the processor unit 3.

Thus the device of the invention enables use of the corresponding method with minimum constraints imposed on the subject, who can of course remain free to move and in a quasi-normal situation, for example at home.

As shown in FIG. 6a, and in addition to an input/output unit I/O for networking this processor unit via the Internet, for example, or via another network, the processor unit 3 includes a central processor unit CPU, working memory RAM, and a hard disk type storage unit for storing the database DBe of cerebral activity analysis data.

The central processor unit 3 further includes a module, formed for example by the program storage modules M₀ and M₁ shown in FIG. 6a, for computing the locations of the set of neuroelectric generators from the positions of the electrodes and from three-dimensional image of the brain acquired from the resources 2.

The computation module can consist of the modules M₀ and M1, the module MO being dedicated to computing the inverse problem to execute the step B₀ of FIG. 3, for example, with the module M₁ being dedicated to executing the meshing operation, i.e. the step B₁ represented in FIG. 3, for example on the basis of the successive sections {Cₖ}₁^{K} obtained from the three-dimensional image acquisition resource 2.

A module M₂ is used to locate the set of neuroelectric generators of the intracerebral neuroelectric signals in accordance with the step B₂ described above and represented in FIG. 3.

Finally, the processing resource 3 advantageously includes a computation module M₃ for discriminating in active areas that include neuroelectric generators, the amount of synchrony that exists between pairs of signals in a plurality of frequency bands, i.e. in accordance with FIGS. 4a, 4b, 4c, and 4d of the drawings.

It is clear in particular that the computation modules M₁, M₁, M₂, and M₃ can advantageously be program modules stored in read-only memory and fetched into the working memory RAM by the central processor unit CPU to execute the corresponding operations.

If so required, the database of reference states representing the dynamic functional image can be stored on the hard disk unit already containing the database DBe, but it is preferably transmitted for storage and use to a particular networked resource that is preferably located in the entity already storing the three-dimensional image of the brain made up of successive sections.

Finally, the device of the invention can advantageously include a resource 4 for stimulating the subject, including a stimulation computer 4₀ for giving the subject either an auditory stimulus by way of earphones 42 or a visual stimulus by displaying on display screens 41 successive images for modifying the subject's state of consciousness, for example psychological test images.

There are many clinical and/or diagnostic applications of the method and the device of the invention.

The method and the device of the invention provide improved location of underlying neuroelectric generators situated within the cerebral volume or on its surface.

The process used has the advantage of accessing functional images with excellent temporal resolution. Moreover, although the surface electrodes measure an instantaneous mix of multiple distributed cerebral activations, the functional imaging effects spatial deconvolution of the information producing a reconstructed temporal course estimate for each position of interest in the brain. By means using the method and the device of the invention, a more refined characterization of cerebral states can be obtained in real time, given the synchronies revealed between the detected neuroelectric generators.

In particular, certain diagnostic results have been demonstrated.

It has been observed that, before a seizure, certain pairs of intracerebral electrodes placed in the vicinity of the periphery of the epileptogenic zone systematically exhibit a significant modification of their synchrony, in particular in the fast frequency bands: α, 8 hertz (Hz) to 12 Hz; β 15 Hz to 30 Hz; and γ 30 Hz to 70 Hz.

Furthermore, these synchronizations have recently received considerable attention because of their possible involvement in large-scale integration phenomena during the cognition process. The corresponding results suggest that the neural populations underlying the epileptogenic area modify their relationship before the seizure with a higher scale dynamic.

These synchronization changes can then lead to dynamic isolation of the epileptogenic focus and they are then liable to provide recurrently a neural population that is easily mobilized by epileptic processes.

The method and the device of the invention then quantify preseizure cerebral activity very precisely. This possibility of anticipating seizures opens up very considerable diagnostic prospects, and where applicable therapeutic prospects, through characterization of the neurobiological modifications that occur during the preseizure phase.

At the clinical level, the possibility of warning the subject and attempting to abort an impending seizure through therapeutic intervention can also be envisaged. In particular, electrical neurostimulation has recently come to light as a promising therapeutic solution for other pathologies, such as Parkinson's disease in particular.

In this light, conservative treatment by electrical stimulation to strengthen or inhibit neural activity can replace mechanical destruction of a predefined cerebral region. The possibility of seizure anticipation through using the method and the device of the invention is the key here, since it answers the question of when to stimulate. The stimulation can be applied when a preseizure is detected with the aim of destabilizing the epileptogenic processes before they become irreversible at the moment of the seizure.

The method and the device of the invention can also drive further development in the field of cognitive intervention. Certain subjects describe their ability to interrupt a seizure when it begins by specific cognitive or motor activities. These phenomena seem likely to be based on destabilization of the epileptic process by the appearance of new electrical activities within the cerebral cortex. Thus modulation of epileptic activity by cognitive synchronization has also been demonstrated using the method and device of the invention.

Finally, other forms of intervention can be envisaged, such as pharmacological intervention, for example, by administering fast-acting anti-epileptic medication such as benzodiazepines. The possibilities of warning and intervention offered by seizure anticipation necessarily imply anticipation in real time, meaning that the computation results and the corresponding detection must be obtained instantaneously and not offline.

The ability to anticipate seizures also improves examinations carried out during the pre-surgical stage of assessing drug-resistant partial epilepsies. In particular, ictal SPECT scans are facilitated by warning the treatment personnel to inject the radioactive tracer at the very beginning of the seizure, or even just before it, so that the epileptogenic focus can be located better. Hospitalization times can then be considerably reduced and imaging system occupation time optimized.

Finally, this example of application to the clinical study of epilepsy can easily be transposed to cognitive activities such as measurement of vigilance, mental workload, or medication/cognition interaction, in particular through modifying the training base consisting of functional images characterizing a cerebral state of the brain of the subject, for example by downloading data.

Consequently, it is clear that the limitations of the earlier techniques stemming from the fact that they assume a linear relationship between stored signals have been removed, by means of the phase synchronization process described above, which would appear to be particularly suitable for measuring the degree of interdependence of the activities of diverse cerebral regions in one or more specific frequency bands.

Thus, and remarkably, neural synchronization in the fast frequency band from 30 Hz to 50 Hz has recently received considerable attention for its possible role in large-scale integration phenomena during cognition and with certain pathologies.

To summarize, the device and the method of the invention locate and quantify in real time interaction between different intracerebral activities, on the basis of electroencephalographic (EEG) signals collected in man, with the aim of characterizing by signature:

1) vigilance, attentiveness, stress, effort, fatigue, etc.;

2) the very short-term evolution of certain pathological states such as epileptic seizures; and

3) the action of drugs and/or medicines, specifically those acting on the central nervous system (CNS).

They can also visualize, classify, and compare these various cerebral states. To be more precise, they can test if a new type of drug or medicine is close to a known drug or medicine, through its signature. In this sense the method and the device of the invention would seem extremely useful for specifying the potential scope of action of a new molecule in man before it is placed on the market.

Finally, the invention covers a computer program product stored on a storage medium for execution by a computer noteworthy in that, upon execution, it executes the method of the invention as described with reference to FIGS. 1b to 4d, and a device for representing a dynamic functional image of the brain as described with reference to FIG. 6a.

1. A method of representing a dynamic functional image of the brain by locating and discriminating intracerebral neuroelectric generators, characterized in that it consists at least:
during a particular recording time, in acquiring a plurality of electrophysiological signals emitted and/or induced by cerebral activity from a plurality of electrodes spread out substantially over the scalp of the cranium protecting the brain, and in digitizing said electrophysiological signals in order to constitute a cerebral activity analysis database;
in locating all the neuroelectric generators in the cerebral volume by acquiring an electronic map of the positions of said electrodes from a three-dimensional image of the brain made up of successive sections, based on segmentation of the cerebral cortex obtained from said three-dimensional image, and from application of the inverse problem to determine the spatial locations of the neuroelectric generators of said intracerebral neuroelectric signals from at least one of the electrophysiological signals, from the electronic map of the location of the electrodes, and from said three-dimensional image of said brain; and
in discriminating in the active areas that include neuroelectric generators the amount of synchrony that exists between pairs of neuroelectric generators in a plurality of frequency bands in order to detect groups of discriminating neural networks and to construct a database of reference states representing said dynamic functional image.

2. A method according to claim 1, characterized in that, for a dynamic functional image acquired during said particular recording time, said method further consists in matching said functional image with one class of functional images from a plurality of classes of functional images, each class of said plurality of classes of functional images characterizing a cerebral state of the brain of the subject.

3. A method according to claim 1 or claim 2, characterized in that the step of acquiring a plurality of electrophysiological signals is effected in real time with a maximum recording delay of less than 100 milliseconds.

4. A method according to any one of claims 1 to 3, characterized in that the recording time is a parameter that can be set over a time range from a minimum period of the order of 20 minutes for storing and representing a functional image of the brain relating to one or more cognitive states, to a period of several days for storing and representing a functional image of the brain relating to one or more provoked or unprovoked functional anomaly states of the brain.

5. A method according to any preceding claim, characterized in that the step of discriminating in active areas the amount of synchrony that exists between pairs of neuroelectric generators is effected during said recording time over a sliding time window of duration that is from 15 milliseconds to 2 seconds to represent a functional image of the brain relating to one or more cognitive states respectively over a time consistency sliding time window of duration that is from 5 seconds to 20 seconds for representing a functional image of the brain relating to one or more provoked or unprovoked functional anomaly states of the brain.

6. A method according to any one of claims 1 to 5, characterized in that the application of the inverse problem to determine the spatial locations of the neuroelectric generators of the intracerebral neuroelectric signals from at least one of the electrophysiological signals, from the electronic map of the location of the electrodes, and from said three-dimensional image of the brain consists at least:
in applying constraints derived from the individual anatomy introduced by segmentation and surface meshing of the parenchyma;
in estimating cortical electric currents by multimode processing of electrophysiological signals;
in computing the positions and functional parameters of said neuroelectric generators in the form of individual electric current sources over the meshing of the cortical surface, an active area including at least one neuroelectric generator.

7. A method according to any one of claims 1 to 6, characterized in that the step of discriminating in said active areas including neuroelectric generators the amount of synchrony that exists between pairs of neuroelectric generators in a frequency band includes statistically evaluating the PLS synchronization between two signals from a pair of neuroelectric generators by means of the circular variance of the phase difference between those signals or the normalized Shannon entropy of that phase difference.

8. A method according to claim 7, characterized in that synchrony is established in synchrony time ranges enabling temporal representation of the activity of said pairs of neuroelectric generators.

9. A dynamic functional image of the brain, characterized in that said dynamic functional image comprises at least:
a three-dimensional image of said brain made up of successive sections each representing an individual image of said brain; and
in at least one individual image, at least one neuroelectric generator of intracerebral neuroelectric signals represented by a marker, each neuroelectric generator being characterized in terms of its position in said individual image, in terms of its electric current density, and in terms of its neuroelectric signal emission direction, all neuroelectric generators of a current individual image adjoining a neuroelectric generator of a preceding and/or subsequent individual image and having substantially the same neuroelectric signal emission direction and synchrony over a particular consistency time constituting a group of neural networks discriminating functional states representing said dynamic functional image of the brain.

10. A functional image according to claim 9, characterized in that, for a functional image relating to a plurality of cognitive states, the temporal consistency time is from 50 milliseconds to 2 seconds.

11. A functional image according to claim 9, characterized in that the temporal consistency time for a functional image relating to one or more provoked or unprovoked functional anomaly states of the brain is from 5 to 20 seconds.

12. A device for representing a dynamic functional image of the brain, characterized in that it comprises at least:
means for acquiring during a particular recording time a plurality of electrophysiological signals emitted and/or induced by cerebral activity from a plurality of electrodes spread out substantially over the scalp of the cranium protecting the brain, and for storing and backing up said electrophysiological signals to constitute a cerebral activity analysis database;
means for acquiring a three-dimensional image of the brain made up of successive sections;
means for computing the locations of the neuroelectric generators of the intracerebral neuroelectric signals from the locations of said electrodes and from said three-dimensional image of said brain, in order to produce segmentation of the cerebral cortex, and for computing the application of the inverse problem;
means for discriminating in the active areas that include neuroelectric generators the amount of synchrony that exists between the electrophysiological signal-neuroelectric generator pairs in a plurality of frequency bands to detect groups of discriminating neural networks and to construct a database of reference states representing said dynamic functional image.

13. A device according to claim 12, characterized in that said means for acquiring a plurality of electrophysiological signals comprise at least a flexible cap fitted with electromagnetic sensors constituting said electrodes and forming a network of sensors pressed onto the scalp of the cranium of the subject.

14. A device according to claim 12 or claim 13, characterized in that it further includes means for visual and/or auditory stimulation of the subject.

15. A computer program product stored on a storage medium for execution by a computer, characterized in that, during execution by a computer, said program product executes the method according to any one of claims 1 to 8 for representing a dynamic functional image of the brain.

16. The use of a method according to any one of claims 1 to 8, a dynamic functional image of the brain according to any one of claims 9 to 11, a device according to any one of claims 12 to 14 for representing a dynamic functional image of the brain, and a computer program product according to claim 15 to characterize by a signature different cerebral states among groups of states relating either to vigilance, attentiveness, stress, effort, fatigue, to the short-term evolution of certain pathological states, or to the action of drugs and/or medicines acting on the central nervous system.
